(19) United States
(12) Patent Application Publication
(10) Pub. No.: US 2022/0226339 A1
(43) Pub. Date: Jul. 21, 2022

(54) INHIBITATION OF ACID LIPASE FOR CANCER THERAPY

(71) Applicant: Heinrich-Heine-Universität Düsseldorf, Düsseldorf (DE)

(72) Inventors: Knud Esser, Köln (DE); Andrea Kulik, München (DE); Tanja Fehm, Düsseldorf (DE)

(21) Appl. No.: 17/617,218

(22) PCT Filed: Jul. 2, 2020
(86) PCT No.: PCT/EP2020/068619

§ 371 Date: Dec. 7, 2021

(30) Foreign Application Priority Data
Jul. 4, 2019 (DE) 10 2019 118 131.3

(51) Int. Cl. A61K 31/5377; G01N 33/50; C12Q 1/44; A61K 31/175; A61K 31/337; A61P 35/00

(52) U.S. Cl. CPC A61K 31/5377; G01N 33/5011; A61P 35/00; A61K 31/175; A61K 31/337; C12Q 1/44

(57) ABSTRACT

The invention relates to new cancer treatment strategies based on the inhibition of acid lipase. In addition, the invention relates to methods for discovering new active substances suitable for cancer therapy.

The invention relates to new cancer treatment strategies based on the inhibition of acid lipase. The invention further relates to methods for finding new active ingredients suitable for cancer therapy.

According to the Robert Koch Institute, 483 000 people developed cancer in Germany in 2013. In 2020, there are expected to be over 500 000 cases. Consequently, malignant tumor diseases are considered to be one of the major challenges of the 21st century. Despite intensive research efforts and new findings in molecular biology, the prognosis of many malignant tumors has, however, improved only insignificantly to date.

Aggressive and rapidly progressing cancers in particular such as triple-negative breast cancer (TNBC) or pancreatic ductal adenocarcinoma can be therapied only with difficulty using hitherto known treatment options. Here, the medical need for innovative oncologic drugs is particularly high.

Present development of innovative cancer drugs involves pursuing the goal of addressing tumor-specific mechanisms in order to be able to control the tumor efficiently and specifically. Here, one aspect of increased focus is the cellular metabolism of cancer cells, which differs in various aspects from healthy cells. This tumor-specific metabolic reprogramming thus leads, for example, to energy production via anaerobic glycolysis even under aerobic conditions, which has long been known as the Warburg effect. More recent studies have revealed further differences, such as, for example, in glutamine metabolism, in the pentose phosphate pathway or in endogenous lipid synthesis.

A metabolic peculiarity especially due to driver mutations in the Ras oncogene of highly aggressive cancers is the greatly increased utilization of both exogenous and endogenous neutral lipids (triglycerides and cholesteryl esters consisting of esterified fatty acids and esterified free cholesterols). Especially in an unfavorable microenvironment with only a small possibility of cellular energy production, which particularly aggressive tumors are commonly exposed to, such as, for example, hypoxic conditions, low nutrient concentrations or other factors causing cell stress, the reduced ability to produce their own lipids means that the efficient uptake and utilization of exogenous lipids and efficient utilization of endogenous lipids represents a crucial survival advantage (T. Petan et al., Molecules 2018, 23, 1941, 1-41).

In addition to increased utilization of exogenous neutral lipids via increased endocytosis of lipoproteins, tumor cells, especially triple-negative tumor cells, exhibit increased utilization of endogenous neutral lipids via increased lipophagy (De Cedron, M. G. & De Molina, A. R. Microtargeting cancer metabolism: Opening new therapeutic windows based on lipid metabolism. Journal of Lipid Research (2016). doi:10.1194/jlr.R061812).

Although the role of cellular neutral lipid and lipid droplets in tumor pathophysiology is still poorly understood and is subject to current research, there are increasing indications of an essential role in cell proliferation, metastasis and anti-apoptotic processes, especially under hypoxia. This metabolism is dysregulated particularly in cells with high stem-cell character such as, for example, triple-negative breast cancer or in tumor stem cells. But also tamoxifen-resistant hormone receptor-positive breast tumors develop higher stem-cell character than tamoxifen-sensitive tumors. The metabolic dysregulations apply to these less differentiated tumor cells with high stem-cell character, tumor stem cells and cells of triple-negative breast cancer.

Furthermore, studies show that the increased utilization of exogenous and endogenous neutral lipids induces epigenetic changes such as, for example, epithelial-mesenchymal transition (EMT). What are particularly crucial for the malignancy of cancer cells are epigenetic changes which determine the stem-cell character of the cancer cells via an increased expression of (tumor) stem cell markers and enable especially tumor cell subpopulations with high stem-cell character (so-called “tumor stem cells”) to survive in so-called “tumor stem cell niches”. Although the role of neutral lipids in this process is still poorly understood, what has been described particularly for “tumor stem cells” is that free fatty acids in particular both from increased uptake of exogenous lipids and via increased lipophagy of endogenous neutral lipids exhibit increased utilization, including via oxidation, and are required for the maintenance of the stem-cell properties of the tumor cells (De Cedron, M. G. & De Molina, A. R. Microtargeting cancer metabolism: Opening new therapeutic windows based on lipid metabolism. Journal of Lipid Research (2016). doi:10.1194/jlr.R061812; Wang, T. et al. JAK/STAT3-Regulated Fatty Acid β-Oxidation Is Critical for Breast Cancer Stem Cell Self-Renewal and Chemoresistance. Cell Metab. (2018). doi:10.1016/j.cmet.2017.11.001). At the same time, the utilization of fatty acids in the tumor microenvironment as well via, for example, macrophages, fibroblasts or stem cells plays a crucial role and can likewise be specifically addressed via the inhibition of acid lipase using specific inhibitors such as, for example, Lalistat and achieve an antitumor effect additional to the effect directed directly against the tumor cells (He, W. et al. MSC-regulated IncRNA MACC1-AS1 promotes stemness and chemoresistance through fatty acid oxidation in gastric cancer. Oncogene (2019). doi:10.1038/s41388-019-0747-0).

Cholesterols are furthermore an essential constituent of “lipid rafts” in cell membranes, which, inter alia, play an important role in many signaling pathways and endocytic processes. There are many studies demonstrating that, for example, “lipid raft”-dependent signaling pathways are substantially involved in malignant cell processes (T. Murai INT J CELL BIOL. 2012; 2012: 763283).

Cells have different receptors via which they accumulate exogenous lipids. Firstly, cholesterols and free fatty acids can be directly taken up into the cell via membrane receptors and stored after esterification. Furthermore, lipoproteins can be endocytosed via receptors of the LDL receptor superfamily (LDL-R, VLDL-R, LRP-1), and the neutral lipids thereof are transported intracellularly to different cellular structures, including “lipid rafts”, as free cholesterols or fatty acids following endosomal hydrolysis and ultimately esterified in the endoplasmic reticulum and stored in lipid droplets. In the case of MDA-MB-231, 436 and PANC-1 cancer cells, it was possible to show that, compared to cell lines of less aggressive forms of cancer such as hormone receptor-positive breast cancer cells, there is a particularly high capability of taking up and accumulating exogenous neutral lipids via the LDL receptor.

The tumor-specific inhibition of the uptake of neutral lipids via receptors of the LDL receptor family thus represents a therapeutic option for substantially improving specifically the treatment of these aggressive forms of cancer and, here in particular, of triple-negative breast cancer cells.

The hitherto pursued options for reduced uptake of neutral lipids from exogenous lipoproteins focus on the inhibition of the LDL receptor itself (D. de Gonzalo-Calvo, BMC Cancer, 2015, 15, 460, 1-13). Current therapeutic approaches to reduce the accumulation of cellular neutral lipids aim at not only the inhibition of esterification (ACAT1), but also at the reduction of the uptake of lipoprotein-derived neutral lipids via the direct inhibition of the LDL receptors. However, these are ubiquitously localized on cell surfaces and cannot be targeted intracellularly by a targeted drug carrier in the cancer cells. Furthermore, the different LDR receptor subtypes (LDLR, VLDLR, LRP-1) are not inhibitable by a single active ingredient.

Besides the inhibition of the utilization of neutral lipids from endocytosed lipoproteins, what can additionally also be inhibited is selective lipid uptake via inhibitors of the scavenger receptor B. Inhibitors such as, for example, BLT-1 (CAS No.: 321673-30-7) can be used for this purpose.

The tumor-specific inhibition of the utilization of endogenous neutral lipids via lipophagy represents a further therapeutic option for substantially improving specifically the treatment of these aggressive forms of cancer and, here in particular, of triple-negative breast cancer cells.

Clinical therapies for the specific inhibition of lipophagy are not available to date. Inhibitors of autophagy, such as, for example, the clinically available chloroquine, may cover the process of lipophagy, but are not specific for this subtype of autophagy.

Lipase inhibitors currently play virtually no role in concepts of cancer therapy. In the case of adipose triglyceride lipase (ATGL), inhibition is a subject of controversial discussion, the enzyme more likely having antitumor activity according to current studies. In the case of the broad-spectrum inhibitor of neutral lipid-hydrolyzing lipases, “Orlistat”, although an antitumor effect has been repeatedly described, said effect is attributed to the inhibition of fatty acid synthase by the inhibitor, primarily in less aggressive tumors. Moreover, a broad inhibition of triglyceride and cholesteryl ester lipases does not appear sensible either, since many cellular lipases mediate the degradation of triglyceride and cholesteryl ester stores and the inhibition thereof would bring about the increased storage of cellular neutral lipid, which is not desirable therapeutically.

There is a need for treatment options for cancers, especially also for aggressive and rapidly progressing cancers.

It is an object of the invention to provide treatment options for cancers, in particular treatment options for aggressive and rapidly progressing cancers, that have advantages over the prior art. It is also an object of the invention to provide methods with which new suitable active ingredients for treatment of such cancers can be found.

This object is achieved by the subject matter of the claims.

It has been found that, surprisingly, inhibitors of acid lipase are suitable for treatment of cancers.

Inhibition of acid lipase reduces or suppresses the endosomal hydrolysis of lipoprotein cholesteryl esters and triglycerides that is essential for further cellular transport to different cellular structures (e.g., cellular “lipid rafts” or mitochondria for beta-oxidation and energy production) and for later storage as neutral lipid esters in lipid droplets. Addressing this endosomal hydrolysis represents the therapeutic approach of the invention for treatment of cancers.

In the intracellular transport of lipoprotein- or lipophagy-derived neutral lipids, the cholesteryl esters and triglycerides are hydrolyzed by acid lipase after being taken up into endosomal compartments and only then further transported intracellularly. Acid lipase hydrolyzes all cholesteryl esters and triglycerides taken up via receptor-mediated endocytosis and lipophagy in the acidic environment of the lysosome. This releases free fatty acids and free cholesterol, which are transported to different cellular structures, where they are available to the cell for further use. In the endoplasmic reticulum, the free cholesterols and fatty acids are esterified and subsequently stored in lipid droplets. Acid lipase thus plays a role in the cellular processing of endocytosed plasma lipoproteins and endogenous lipid droplets and contributes to the homeostatic control of lipid levels in plasma and in the cell.

According to current knowledge, acid lipase is the only cellular enzyme which carries out the hydrolysis presented under paragraph [0022] in the late endosome/lysosome. The invention relates to the specific inhibition of said enzyme as a therapeutic approach for aggressive tumor types in particular, preferably under hypoxic conditions or other cell stress-inducing conditions. The selective inhibition of acid lipase according to the invention specifically addresses the cellular utilization of lipoprotein- and lipophagy-derived free cholesterols and fatty acids (Zhang, H. Lysosomal acid lipase and lipid metabolism: New mechanisms, new questions, and new therapies. Current Opinion in Lipidology (2018). doi:10.1097/MOL.0000000000000507).

The pharmacological inhibition of endosomal acid lipase according to the invention via, for example, Lalistat, addresses, for the first time and simultaneously, two pathomechanisms present especially in TNBC: the increased utilization in the cancer cells of (I) exogenous neutral lipids via increased endocytosis and of (II) endogenous neutral lipids via increased lipophagy. Firstly, the neutral lipids are used in the cancer cells to cover the high energy requirement via oxidation and, secondly, the oxidation of fatty acids maintains the high stem-cell character, particularly in breast cancer tumor stem cells, via a pathomechanism not yet understood in many steps (Wang, T. et al. JAK/STAT3-Regulated Fatty Acid (3-Oxidation Is Critical for Breast Cancer Stem Cell Self-Renewal and Chemoresistance. Cell Metab. (2018). doi:10.1016/j.cmet.2017.11.001). Lalistat can be transported into the cancer cells in a targeted manner via pharmaceutical carriers without reaching peripheral healthy tissue. This concept can thus be easily integrated into personalized, targeted medicine, especially in the case of hardly therapiable aggressive tumors having mutations in the RAS oncogene.

Moreover, the pharmacological inhibition of endosomal acid lipase according to the invention via, for example, Lalistat covers, as single active ingredient, the different LDL receptor subtypes (LDLR, VLDLR, LRP-1) together.

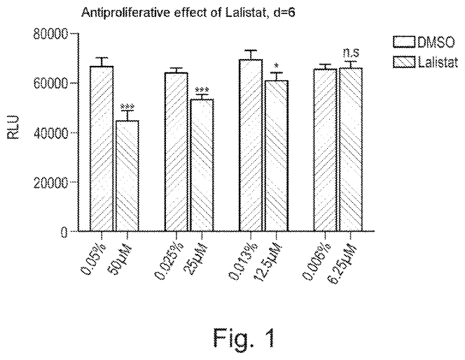

FIG. 1 shows the antiproliferative effect of the acid lipase inhibitor Lalistat 2 on the cell growth of MDA-MB-231 cancer cells in the range of non-cell-toxic concentrations. Cell proliferation was ascertained using Cell-TiterGlo®.

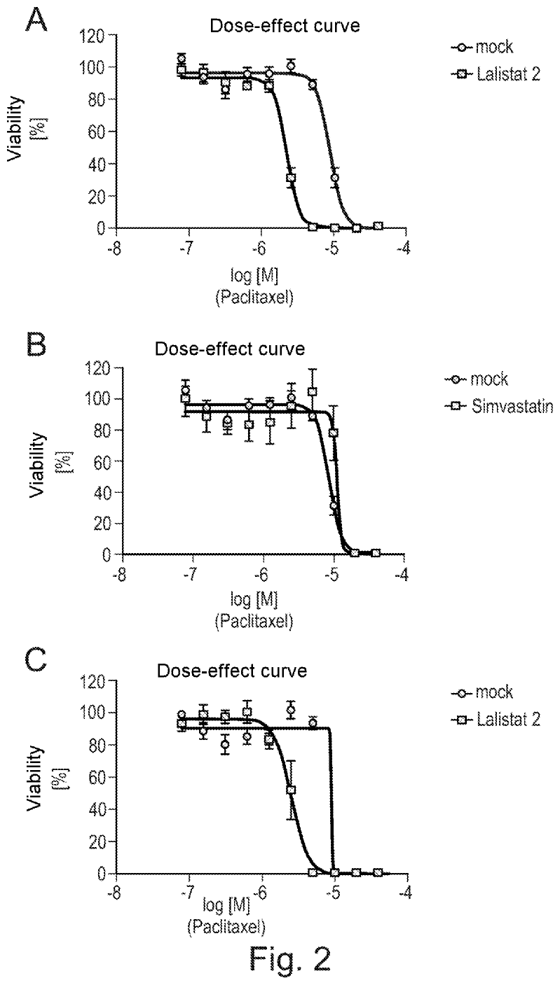

FIG. 2 shows the effects of acid lipase-inhibiting substances on the toxicity of paclitaxel in TNBC (A, B) MDA-MB-231 cancer cells and in pancreatic ductal adenocarcinoma (C) PANC-1 cancer cells.

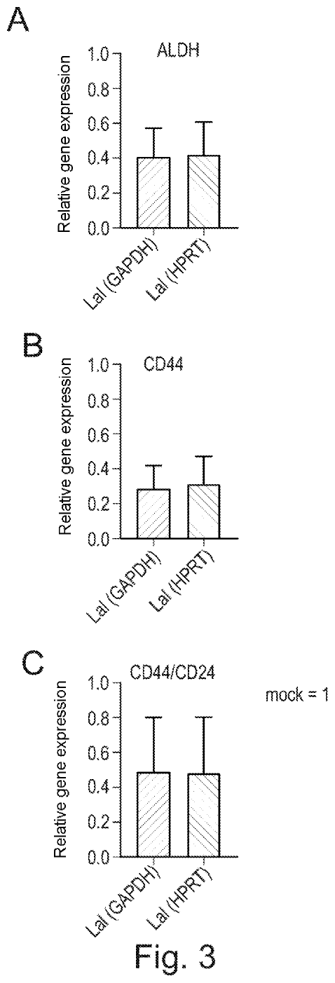

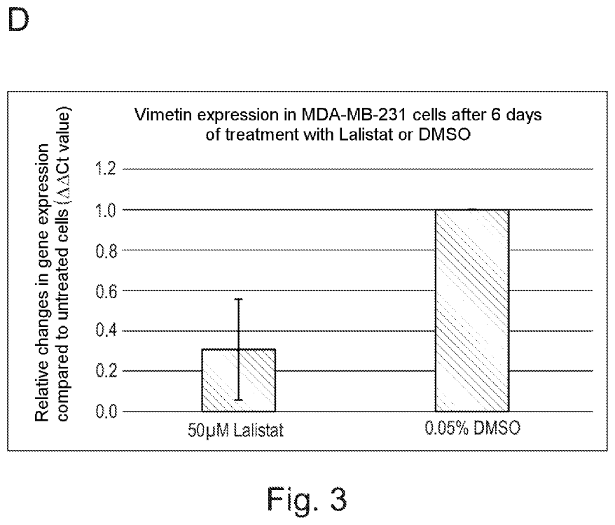

FIG. 3 shows the relative change in expression of tumor stem cell markers and vimentin in MDA-MB-231 cancer cells after 6 days of treatment with the acid lipase inhibitor Lalistat 2.

A first aspect of the invention relates to a method for finding inhibitors of acid lipase that are suitable for treatment of cancers, preferably for adjuvant or possibly also monotherapeutic treatment, the method comprising the following steps:
(a) providing an inhibitor of acid lipase;
(b) incubating cancer cells of a cancer cell line with the inhibitor of acid lipase and ascertaining a property of the cancer cells;
(c) incubating cancer cells of the same cancer cell line as in step (b) in the absence of the inhibitor of acid lipase and ascertaining the same property of the cancer cells as in step (b) under the same conditions as in step (b);
(d) comparing the ascertained property of the cancer cells according to steps (b) and (c).

The suitability of the inhibitors of acid lipase can concern the monotherapeutic treatment of cancers, meaning that the method according to the invention is used for finding inhibitors which allow therapy just by themselves, i.e., without interaction with other substances or cells. According to this embodiment, the absence of the inhibitor of acid lipase is preferably the only difference between step (c) and step (b). According to this embodiment, steps (b) and (c) are each preferably carried out in the absence of (i) cell-toxic substances, (ii) substances having an antitumor effect and (iii) immune cells directed against cancer cells.

Unless otherwise expressly stated, the term “substance” refers to both cell-toxic substances and substances having an antitumor effect in what follows for the purpose of describing the invention.

In a preferred embodiment, the method according to the invention is used for finding inhibitors of acid lipase that are suitable for adjuvant treatment of cancers, wherein step (b) is carried out in the presence of (i) a cell-toxic substance, (ii) a substance having an antitumor effect or (iii) immune cells directed against cancer cells; and wherein step (c) is carried out in the presence of the same substance or the same immune cells as in step (b).

The order of steps (a) to (d) of the method according to the invention is not fixed, though step (a) will naturally be carried out at the start and step (d) will naturally be carried out toward the end. Steps (b) and (c) can be carried out successively in any order, simultaneously or semi-simultaneously.

In step (a) of the method according to the invention, an inhibitor of acid lipase is provided. It is preferably an inhibitor which is selective for acid lipase, i.e., which does not inhibit the enzymatic activity of other lipases such as, for example, lipoprotein lipase (LPL), hepatic lipase, monoacylglycerol lipase (MAGL), diacylglycerol lipase (DAGL) and lipid droplets-associated lipases or inhibits said enzymatic activity only to a substantially lesser extent. Furthermore, in addition to other lipases, the inhibitor should not show any activity toward further serine hydrolases such as, for example, acetylcholinesterase (AChE).

Inhibitors and selective inhibitors of acid lipase are known and, inter alia, also commercially available. Two inhibitors of acid lipase that are particularly preferred according to the invention are Lalistat 1 (CAS No. 501104-16-1, 4-(piperidin-1-yl)-1,2,5-thiadiazol-3-yl morpholine-4-carboxylate)

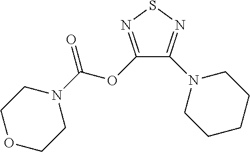 [SMILES: O=C(Oc1nsnc1N1CCCCC1)N1CCOCC1]

and
Lalistat 2 (CAS No. 1234569-09-5, 4-(piperidin-1-yl)-1,2,5-thiadiazol-3-yl piperidine-1-carboxylate)

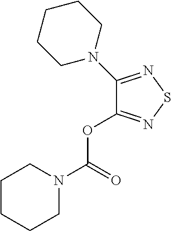 [SMILES: O=C(Oc1nsnc1N1CCCCC1)N1CCCCC1]

For the purposes of the invention, “Lalistat” is preferably understood to mean Lalistat 2 or Lalistat 1 or a mixture of Lalistat 1 and Lalistat 2 in any mixing ratio.

For the purposes of the invention, “acid lipase” is preferably understood to mean human intracellular endosomal acid lipase (lysosomal acid lipase, LAL, EC 3.1.1.13).

In steps (b) and (c) of the method according to the invention, cancer cells of a cancer cell line are used. Suitable cancer cell lines for a wide variety of cancers are commercially available. A person skilled in the art will recognize that different cancer cell lines can be used in the method according to the invention depending on the cancer addressed.

The cancer cell line is preferably selected from human cell lines originating from patients suffering from a cancer selected from the group consisting of uveal melanoma, basal-cell carcinoma, B-cell lymphoma, chondrosarcoma, CHRPE, desmoid tumors, small intestine carcinoma, embryonal tumours, endometrial carcinoma, ependymoma, Fanconi anemia/medulloblastoma, thyroid follicular cell tumors, gastrointestinal carcinomas, gastrointestinal stromal tumors, glioblastoma, glioma, gonadoblastoma, hemangioblastomas of the retina and the CNS, breast hamartomas, hamartomas of the gastrointestinal tract, bladder carcinoma, skin carcinoma, skin tumors, hepatobiliary carcinomas, hepatoblastoma, hepatoma, hereditary leiomyomatosis, brain tumours, pituitary adenoma, keratoacanthomas, colon carcinoma, cutaneous melanoma, larynx carcinoma, leiomyosarcoma, skin leiomyoma, leukemia, Lhermitte-Duclos disease, liposarcoma, lymphoma, stomach and small intestine carcinomas, stomach carcinoma, diffuse type, malignant tumors of the gastrointestinal tract, malignant tumors of the urogenital tract, malignant melanoma, malignomas of the hematopoietic system, mammary carcinoma, mammary fibroadenoma, medullary thyroid carcinomas, medulloblastoma, medulloblastomas and other CNS tumors, melanoma, melanocytic skin lesions, meningioma, mesothelioma, multiple bilateral renal angiomyolipoma, myelodysplasia, myocardial rhabdomyoma, myxoid subcutaneous tumors, adrenocortical carcinoma, parathyroid adenomas, parathyroid hyperplasia, neurofibrosarcomas, renal pelvis carcinoma, kidney tumors (oncocytoma, chromophobe renal cell carcinoma, hybrid tumors), renal cell carcinoma, renal cell carcinoma-associated renal cell carcinoma, odontogenic keratocysts, optic gliomas, osteogenic sarcoma, osteoma, osteosarcoma, osteosarcoma, ovarian carcinoma, pancreatic islet cell tumors, pancreatic carcinoma, papillary renal cell carcinoma, papillary thyroid carcinoma, paraganglioma, pheochromocytoma, pheochromocytomas, squamous cell carcinoma, squamous cell carcinoma of the skin, squamous cell carcinomas, choroid plexus tumors, primary adrenocortical nodular hyperplasia, primitive neuroectodermal tumors, prostate carcinoma, retinoblastoma, rhabdoid tumors, rhabdomyosarcoma, giant cell astrocytoma, sarcoma/osteosarcoma, thyroid carcinoma, schwannoma, sebaceous adenomas, sebaceous epitheliomas, sebaceous carcinoma, testicular Sertoli cell tumor, testicular carcinoma, trichilemmomas of the skin, ureter carcinoma, uterine leiomyomas, vestibular schwannomas, atrial myxoma, soft tissue sarcoma, Wilms tumor and tongue carcinoma; preferably breast cancer, preferably triple-negative breast cancer, and pancreatic carcinoma.

The cancer cell line is preferably selected from the group consisting of MDA-MB-231 cancer cells, MDA-MB-436 cancer cells and PANC-1 cancer cells.

The number of cancer cells which are used in step (b) and in step (c) of the method according to the invention is not limited. According to the invention, use is made of at least one individual cancer cell in each case, but preferably a defined multiplicity of cancer cells.

In a preferred embodiment, if the method according to the invention is used for finding inhibitors of acid lipase that are suitable for adjuvant treatment of cancers, what is preferably carried out in steps (b) and (c) of the method according to the invention is incubation of the cancer cells (i) with a cell-toxic substance, (ii) with a substance having an antitumor effect or (iii) with immune cells directed against cancer cells, additionally in the presence of the inhibitor of acid lipase in step (b) but in the absence of the inhibitor of acid lipase in step (c). To this end, the substance or the immune cells are preferably provided in a medium and brought together with the cancer cells. Suitable concentrations of the substance or the immune cells depend on their respective specific efficacy and can be ascertained by simple routine experiments.

Suitable incubation conditions such as temperature, medium, pH, etc. are known to a person skilled in the art and sometimes depend on the chosen cancer cell line. A medium preferred according to the invention is RPMI medium (developed by the Roswell Park Memorial Institute), preferably RPMI-1640, which preferably contains 15% FCS (fetal calf serum). Such media are known to a person skilled in the art and are commercially available.

According to the invention, the duration of incubation is preferably chosen such that a potential effect of (i) the cell-toxic substance, (ii) the substance having an antitumor effect or (iii) the immune cells directed against cancer cells can be observed. Depending on the chosen cancer cell line and chosen substance or immune cell, the suitable duration of incubation can range from a few minutes to several hours or days. Suitable periods of time can be ascertained by simple routine tests. In preferred embodiments, the duration of incubation is 1 to 12 days, more preferably 3 to 9 days, particularly preferably 6 days.

In a preferred embodiment, the cancer cells are first seeded and it is only after a certain period of time, preferably only after 12 to 36 hours and particularly preferably only after 24 hours, that they are incubated (i) with the cell-toxic substance, (ii) with the substance having an antitumor effect or (iii) with the immune cells directed against cancer cells.

In a preferred embodiment, preincubation is carried out for preferably 1 to 6 days, preferably 3 days, before addition of (i) the toxic substance, (ii) the substance having an antitumor effect or (iii) the immune cells directed against cancer cells, but after addition of the inhibitor of acid lipase.

In steps (b) and (c) of the method according to the invention, incubation is followed by ascertaining a property of the cancer cells. This entails pursuing the goal of ascertaining different properties of the cancer cells that are attributable to the differing incubation in steps (b) and (c), more particularly to the presence of the inhibitor of acid lipase in step (b) of the method according to the invention, either in the absence (monotherapy) or in the presence (adjuvant therapy) of (i) the cell-toxic substance, (ii) the substance having an antitumor effect or (iii) the immune cells directed against cancer cells.

A person skilled in the art will recognize that fundamentally different properties of cancer cells are suitable for ascertaining the effect of incubation in the presence or absence of certain substances.

The property of the cancer cells that is ascertained is preferably the survivability thereof This is especially preferred if (i) the cell-toxic substance, (ii) the substance having an antitumor effect or (iii) the immune cells directed against cancer cells are usually suitable for this purpose or have the goal of causing the death of the cancer cells, for example by apoptosis.

Methods for ascertaining the survivability (viability) of cancer cells are known to a person skilled in the art and suitable assays are commercially available. In a preferred embodiment, the survivability of the cancer cells is ascertained according to the invention with the aid of the assay CellTiter-Glo® (Promega).

The assessment of the property of the cancer cells after incubation, for example the assessment of survivability, can be performed individually and subjectively by the experimenter, for example on the basis of a color reaction.

In a preferred embodiment of the method according to the invention, the ascertainment of property comprises quantitative measurement, preferably of the cellular ATP concentration via the measurement variable luminescence. Instruments preferably suitable according to the invention, such as, for example, spectrometers, are used for this purpose.

The (i) cell-toxic substance preferably used in steps (b) and (c) of the method according to the invention is not defined. It is preferably a known cell-toxic substance which has preferably been authorized for clinical applications and is preferably also already used for cancer treatments. In preferred embodiments, the cell-toxic substance is selected from the group consisting of taxol, docetaxel, cisplatin, carboplatin, cytochalasin B, gramicidin D, ethidium bromide, emetine, mitomycin, etoposide, teniposide, vincristine, vinblastine, colchicine, doxorubicin, daunorubicin, dihydroxyanthracenedione, mitoxantrone, mithramycin, actinomycin, d, 1-dihydrotestosterone, glucocorticoids, procaine, tetracaine, lidocaine, propranolol and puromycin; preferably taxol.

The (ii) substance having an antitumor effect preferably used in steps (b) and (c) of the method according to the invention is not defined. It is preferably a known substance having an antitumor effect which has preferably been authorized for clinical applications and is preferably also already used for cancer treatments. In preferred embodiments, the substance having an antitumor effect is selected from the group consisting of
targeted active antitumor ingredients such as, for example, tyrosine kinase inhibitors, preferably selected from the group consisting of EGFR inhibitors, BRAF inhibitors, PIK3 inhibitors, PIK3/mTOR inhibitors and HER-2 inhibitors; examples preferred according to the invention are:
   EGFR inhibitors: for example erlotinib, gefitinib, lapatinib, cetuximab, neratinib, osimertinib, panitumumab, vandetanib, necitumumab, dacomitinib;
   BRAF inhibitors: for example vemurafenib, dabrafenib, sorafenib, encorafenib;
   PIK3 inhibitors: for example buparlisib, idelalisib, copanlisib, duvelisib, alpelisib, taselisib, perifosine, umbralisib;
   dual PIK3/mTOR inhibitors: for example dactolisib, apitolisib, gedatolisib, bimiralisib, P7170, SF-1126, GDC-0084, LY3023414;
   HER-2 inhibitors: for example trastuzumab, lapatinib, neratinib, ado-trastuzumab emtansine, pertuzumab, dacomitinib;
active ingredients of anti-hormone therapy, preferably selected from antiestrogens, aromatase inhibitors and GnRH analogs; examples preferred according to the invention are:
   antiestrogens: for example tamoxifen, fulvestrant;
   aromatase inhibitors: anastrozole, letrozole;
   GnRH analogs: leuprorelin, goserelin; or
immunostimulatory antibodies, such as, for example, antibodies directed against PD-L1, antibodies directed against PD-1 or antibodies directed against CTLA-4. Preference is given here to, optionally, coincubation with (iii) immune cells directed against the cancer cells.

The (iii) immune cells directed against cancer cells that are preferably used in steps (b) and (c) of the method according to the invention are not defined. They are preferably therapeutically modified immune cells which are preferably authorized for clinical applications and preferably also already used for cancer treatment. In preferred embodiments, the immune cells directed against cancer cells are modified cytotoxic T cells. Here, modifications can be present, for example, on the T-cell receptor for recognition of cancer-specific antigens or CART cells can be used.

According to the invention, what can be used are individual species (i), (ii) or (iii), or any combination, more particularly
individual cell-toxic substances or combinations of multiple cell-toxic substances,
single substances having an antitumor effect or multiple substances having an antitumor effect,
single type of immune cells directed against cancer cells or different types of immune cells directed against cancer cells,
a cell-toxic substance in combination with a substance having an antitumor effect,
a cell-toxic substance in combination with immune cells directed against cancer cells, or
a substance having an antitumor effect in combination with immune cells directed against cancer cells.

The property of the cancer cells is preferably ascertained at a plurality of concentrations of (i) the cell-toxic substance, (ii) the substance having an antitumor effect or (iii) the immune cells directed against cancer cells in step (b) and in step (c) of the method according to the invention. On the basis of the results for various concentrations, what is preferably subsequently ascertained is an IC₅₀ value as that concentration of (i) the cell-toxic substance, (ii) the substance having an antitumor effect or (iii) the immune cells directed against cancer cells at which 50% of the cancer cells have the ascertained property. A comparison of the various ICso values for various inhibitors of acid lipase then allows an assessment of the possible suitability of the inhibitors of acid lipase for the treatment of the cancer addressed as per the cancer cell line. The smaller the ICso value, the better the possible suitability of the inhibitor. A person skilled in the art will recognize that a wide range of other factors not captured by the IC₅₀ value are relevant to actual clinical application.

A major difference between step (b) and step (c) of the method according to the invention is that step (b) is carried out in the presence of the inhibitor of acid lipase, whereas step (c) is carried out in the absence thereof.

In a preferred embodiment of the method according to the invention, the absence of the inhibitor of acid lipase is the only difference between step (c) and step (b).

In another preferred embodiment of the method according to the invention, a further difference between step (c) and step (b), in addition to the absence of the inhibitor of acid lipase, is that step (c) is carried out in the presence of another inhibitor. In this case, the other inhibitor in step (c) is preferably an HMG-CoA reductase inhibitor; preferably a statin; particularly preferred simvastatin.

In a preferred embodiment of the method according to the invention, the property of the cancer cells is ascertained at a plurality of concentrations of the inhibitor of acid lipase in step (b).

The method according to the invention is suitable for testing known inhibitors of acid lipase with respect to the suitability thereof for treatment of a cancer, specifically as monotherapy or as adjuvant therapy, together (i) with a cell-toxic substance, (ii) with a substance having an antitumor effect or (iii) with immune cells directed against cancer cells if applicable. The specific cancer is determined by the choice of cancer cell line used.

In a preferred embodiment, the method according to the invention comprises, for provision of the inhibitor of acid lipase in step (a), the upstream screening of a library of substances or other collection of multiple test substances with respect to their possible inhibitory effect on the enzymatic activity of acid lipase. Said upstream screening is preferably based on a comparatively simple in vitro assay which does not require cancer cell lines. Said upstream screening is preferably designed as HTS (high-throughput screening) and may be effected in an automated or semiautomated manner.

In this case, step (a) preferably comprises the substeps of
(a₁) providing multiple test substances;
(a₂) screening the test substances for their inhibitory effect on the enzymatic activity of acid lipase;
(a₃) selecting at least one screened test substance, the inhibitory effect of which is stronger than the inhibitory effect of at least one other screened test substance, and providing this selected test substance as an inhibitor of acid lipase.

Preferably, step (a₂) comprises respectively measuring, separately from one another under otherwise identical conditions, the rate of conversion of a substrate of acid lipase under enzymatic catalysis by acid lipase in the presence of the individual test substances.

Suitable reaction conditions for the conversion (medium, temperature, pH, cofactors, etc.) are known to a person skilled in the art and can be ascertained by customary routine experiments. Suitable substrates for the conversion are likewise known to a person skilled in the art. A substrate preferred according to the invention is the ester of palmitic acid with 4-propyl-8-methyl-7-hydroxycoumarin (P-PMHC), the conversion of which can be measured fluorimetrically (see S. Masi et al., Clin Chem. 2018 Apr; 64(4): 690-696). Alternatively, pyrenemethyl laurate is preferred (cf. A. Negre et al., Enzyme 1989; 42(2):110-7).

A further aspect of the invention relates to the use of an inhibitor of acid lipase, preferably Lalistat or one of its physiologically acceptable salts, for production of a drug for treatment of a cancer, preferably for treatment of cancer cells having a high tumor-stem-cell character, preferably for treatment of “tumor stem cells”, preferably for adjuvant treatment of a cancer, preferably under hypoxic or other cell stress-inducing conditions. A further aspect of the invention relates to a method for treating a cancer, preferably for treating cancer cells having a high tumor-stem-cell character, preferably for treating “tumor stem cells”, preferably for adjuvant treatment of a cancer, preferably under hypoxic or other cell stress-inducing conditions, comprising administering a drug containing an inhibitor of acid lipase, preferably Lalistat or one of its physiologically acceptable salts, to a patient. A further aspect of the invention relates to an inhibitor of acid lipase, preferably Lalistat or one of its physiologically acceptable salts, for use in the treatment of a cancer, preferably for treatment of cancer cells having a high tumor-stem-cell character, preferably for treatment of “tumor stem cells”, preferably for use in an adjuvant treatment of a cancer, preferably under hypoxic or other cell stress-inducing conditions. Preferably, (i) the treatment increases the apoptosis inducibility of the cancer cells, and/or (ii) the treatment reduces the proliferation of the cancer cells, and/or (iii) the treatment reduces the mesenchymal character and/or stem-cell character of the cancer cells, and/or (iv) the treatment increases the immunogenicity of the cancer cells, and/or (v) the treatment reduces or prevents the metastasis of the cancer cells and/or kills metastases-forming circulating tumor cells and/or cell clusters and/or prevents the formation of metastases therefrom, and/or (vi) the cancer cells to be treated are tumor stem cells.

The inhibitor is preferably a selective inhibitor of acid lipase, as has already been described above and to which reference is made in full.

The treatment is preferably effected as an adjuvant treatment in addition to a treatment (i) with a cell-toxic substance, (ii) with a substance having an antitumor effect or (iii) with immune cells directed against cancer cells. The substances or immune cells are preferably substances or immune cells as have already described above and to which reference is made in full.

In a preferred embodiment, the drug contains the inhibitor of acid lipase, preferably Lalistat or one of its physiologically acceptable salts, and also (i) the cell-toxic substance, (ii) the substance having an antitumor effect or (iii) the immune cells directed against cancer cells. In this case, it is then necessary for the administration to take place simultaneously via a common route of administration, preferably intravenously.

In another preferred embodiment, the drug contains the inhibitor of acid lipase, preferably Lalistat or one of its physiologically acceptable salts, but (i) no cell-toxic substance, (ii) no substance having an antitumor effect and (iii) no immune cells directed against cancer cells. In said embodiment, (i) the cell-toxic substance, (ii) the substance having an antitumor effect or (iii) the immune cells directed against cancer cells is/are possibly contained in another drug. In this case, the two drugs can be administered simultaneously or at different times and via the same route of administration or via different routes of administration.

The appropriate dosage of (i) the cell-toxic substance, (ii) the substance having an antitumor effect or (iii) the immune cells directed against cancer cells and of the inhibitor of acid lipase can be ascertained independently of one another via dose-finding studies, which are known to a person skilled in the art. The dose required may depend on multiple factors such as severity of the disease, height and weight of the patient, age of the patient, route of administration, etc.

Administration can take place several times a day, once a day, once a week or else just once altogether. The frequency of administration may likewise depend on multiple factors such as severity of the disease, height and weight of the patient, age of the patient, route of administration, etc.

The cancer treated is preferably selected from the group consisting of uveal melanoma, basal-cell carcinoma, B-cell lymphoma, chondrosarcoma, CHRPE, desmoid tumors, small intestine carcinoma, embryonal tumours, endometrial carcinoma, ependymoma, Fanconi anemia/medulloblastoma, thyroid follicular cell tumors, gastrointestinal carcinomas, gastrointestinal stromal tumors, glioblastoma, glioma, gonadoblastoma, hemangioblastomas of the retina and the CNS, breast hamartomas, hamartomas of the gastrointestinal tract, bladder carcinoma, skin carcinoma, skin tumors, hepatobiliary carcinomas, hepatoblastoma, hepatoma, hereditary leiomyomatosis, brain tumours, pituitary adenoma, keratoacanthomas, colon carcinoma, cutaneous melanoma, larynx carcinoma, leiomyosarcoma, skin leiomyoma, leukemia, Lhermitte-Duclos disease, liposarcoma, lymphoma, stomach and small intestine carcinomas, stomach carcinoma, diffuse type, malignant tumors of the gastrointestinal tract, malignant tumors of the urogenital tract, malignant melanoma, malignomas of the hematopoietic system, mammary carcinoma, mammary fibroadenoma, medullary thyroid carcinomas, medulloblastoma, medulloblastomas and other CNS tumors, melanoma, melanocytic skin lesions, meningioma, mesothelioma, multiple bilateral renal angiomyolipoma, myelodysplasia, myocardial rhabdomyoma, myxoid subcutaneous tumors, adrenocortical carcinoma, parathyroid adenomas, parathyroid hyperplasia, neurofibrosarcomas, renal pelvis carcinoma, kidney tumors (oncocytoma, chromophobe renal cell carcinoma, hybrid tumors), renal cell carcinoma, renal cell carcinoma-associated renal cell carcinoma, odontogenic keratocysts, optic gliomas, osteogenic sarcoma, osteoma, osteosarcoma, osteosarcoma, ovarian carcinoma, pancreatic islet cell tumors, pancreatic carcinoma, papillary renal cell carcinoma, papillary thyroid carcinoma, paraganglioma, pheochromocytoma, pheochromocytomas, squamous cell carcinoma, squamous cell carcinoma of the skin, squamous cell carcinomas, choroid plexus tumors, primary adrenocortical nodular hyperplasia, primitive neuroectodermal tumors, prostate carcinoma, retinoblastoma, rhabdoid tumors, rhabdomyosarcoma, giant cell astrocytoma, sarcoma/osteosarcoma, thyroid carcinoma, schwannoma, sebaceous adenomas, sebaceous epitheliomas, sebaceous carcinoma, testicular Sertoli cell tumor, testicular carcinoma, trichilemmomas of the skin, ureter carcinoma, uterine leiomyomas, vestibular schwannomas, atrial myxoma, soft tissue sarcoma, Wilms tumor and tongue carcinoma; preferably breast cancer (mammary carcinoma), preferably triple-negative breast cancer (TNBC), and pancreatic carcinoma.

A further aspect of the invention relates to a pharmaceutical composition comprising an inhibitor of acid lipase, preferably Lalistat or one of its physiologically acceptable salts, and also (i) a cell-toxic substance, (ii) a substance having an antitumor effect or (iii) immune cells directed against cancer cells.

Preferred inhibitors of acid lipase and preferred (i) cell-toxic substances, (ii) substances having an antitumor effect or (iii) immune cells directed against cancer cells are those as have already described above and to which reference is made in full.

The composition according to the invention is preferably prepared for intravenous administration. The composition according to the invention is preferably prepared for treatment of a cancer, particularly preferably prepared for treatment of triple-negative mammary carcinoma or tamoxifen- or hormone therapy-resistant hormone receptor-positive breast cancer. A further preferred embodiment relates to the composition according to the invention for use in the treatment of a cancer, particularly preferably for use in the treatment of triple-negative mammary carcinoma or tamoxifen-resistant hormone receptor-positive breast cancer. A further preferred embodiment relates to the use of the composition according to the invention for production of a drug for treatment of a cancer, preferably for production of a drug for treatment of triple-negative mammary carcinoma or tamoxifen-resistant hormone receptor-positive breast cancer.

Preferred embodiments 1 to 27 of the invention are: 1. A method for finding inhibitors of acid lipase that are suitable for treatment of cancers, comprising the steps of (a) providing an inhibitor of acid lipase; (b) incubating cancer cells of a cancer cell line with the inhibitor of acid lipase and ascertaining a property of the cancer cells; (c) incubating cancer cells of the same cancer cell line as in step (b) in the absence of the inhibitor of acid lipase and ascertaining the same property of the cancer cells as in step (b) under the same conditions as in step (b); (d) comparing the ascertained property of the cancer cells according to steps (b) and (c). 2. The method according to embodiment 1 for finding inhibitors of acid lipase that are suitable for adjuvant treatment of cancers, wherein step (b) is carried out in the presence of (i) a cell-toxic substance, (ii) a substance having an antitumor effect or (iii) immune cells directed against cancer cells; and wherein step (c) is carried out in the presence of the same substance or the same immune cells as in step (b). 3. The method according to embodiment 2, wherein the property of the cancer cells is ascertained at a plurality of concentrations of the substance or the immune cells in step (b) and in step (c). 4. The method according to any of the preceding embodiments, wherein the property of the cancer cells is ascertained at a plurality of concentrations of the inhibitor of acid lipase in step (b). 5. The method according to any of the preceding embodiments, wherein the absence of the inhibitor of acid lipase is the only difference between step (c) and step (b). 6. The method according to any of embodiments 1 to 4, wherein a further difference between step (c) and step (b), in addition to the absence of the inhibitor of acid lipase, is that step (c) is carried out in the presence of another inhibitor. 7. The method according to embodiment 6, wherein the other inhibitor in step (c) is an HMG-CoA reductase inhibitor; preferably a statin; particularly preferably simvastatin. 8. The method according to any of the preceding embodiments, wherein the property of the cancer cells that is ascertained is the survivability thereof 9. The method according to any of the preceding embodiments, wherein the ascertainment of property comprises quantitative measurement of a measurement variable. 10. The method according to any of embodiments 2 to 9, wherein the (i) cell-toxic substance is selected from the group consisting of taxol, docetaxel, cisplatin, carboplatin, cytochalasin B, gramicidin D, ethidium bromide, emetine, mitomycin, etoposide, teniposide, vincristine, vinblastine, colchicine, doxorubicin, daunorubicin, dihydroxyanthracenedione, mitoxantrone, mithramycin, actinomycin, d,l-dihydrotestosterone, glucocorticoids, procaine, tetracaine, lidocaine, propranolol and puromycin; preferably taxol. 11. The method according to any of embodiments 2 to 9, wherein the (ii) substance having an antitumor effect is selected from the group consisting of targeted active antitumor ingredients, active ingredients of anti-hormone therapy and immunostimulatory antibodies. 12. The method according to any of embodiments 2 to 9, wherein the (iii) immune cells directed against cancer cells are modified cytotoxic T cells. 13. The method according to any of the preceding embodiments, wherein the cancer cell line is selected from the group consisting of MDA-MB-231 cancer cells, MDA-MB-436 cancer cells and PANC-1 cancer cells. 14. The method according to any of the preceding embodiments, wherein step (a) comprises the substeps of (a1) providing multiple test substances; (a2) screening the test substances for their inhibitory effect on the enzymatic activity of acid lipase; (a3) selecting at least one screened test substance, the inhibitory effect of which is stronger than the inhibitory effect of at least one other screened test substance, and providing this selected test substance as an inhibitor of acid lipase. 15. The method according to embodiment 14, wherein step (a2) comprises respectively measuring, separately from one another under otherwise identical conditions, the rate of conversion of a substrate of acid lipase under enzymatic catalysis by acid lipase in the presence of the individual test substances. 16. The use of an inhibitor of acid lipase, preferably Lalistat or one of its physiologically acceptable salts, for production of a drug for treatment of a cancer. 17. The use according to embodiment 16, wherein the treatment is effected as an adjuvant treatment in addition to a treatment (i) with a cell-toxic substance, (ii) with a substance having an antitumor effect or (iii) with immune cells directed against cancer cells. 18. The use according to embodiment 17, wherein the drug contains the inhibitor of acid lipase, preferably Lalistat or one of its physiologically acceptable salts, and also (i) the cell-toxic substance, (ii) the substance having an antitumor effect or (iii) the immune cells directed against cancer cells. 19. The use according to embodiment 17, wherein the drug contains the inhibitor of acid lipase, preferably Lalistat or one of its physiologically acceptable salts, and wherein (i) the cell-toxic substance, (ii) the substance having an antitumor effect or (iii) the immune cells directed against cancer cells are contained in another drug. 20. The use according to any of embodiments 17 to 19, wherein the (i) cell-toxic substance is selected from the group consisting of taxol, docetaxel, cisplatin, carboplatin, cytochalasin B, gramicidin D, ethidium bromide, emetine, mitomycin, etoposide, teniposide, vincristine, vinblastine, colchicine, doxorubicin, daunorubicin, dihydroxyanthracenedione, mitoxantrone, mithramycin, actinomycin, d,1-dihydrotestosterone, glucocorticoids, procaine, tetracaine, lidocaine, propranolol and puromycin; preferably taxol. 21. The use according to any of embodiments 17 to 19, wherein the (ii) substance having an antitumor effect is selected from the group consisting of targeted active antitumor ingredients, active ingredients of anti-hormone therapy and immunostimulatory antibodies. 22. The use according to any of embodiments 17 to 19, wherein the (iii) immune cells directed against cancer cells are modified cytotoxic T cells. 23. The use according to any of embodiments 16 to 22, wherein the cancer is selected from the group consisting of uveal melanoma, basal-cell carcinoma, B-cell lymphoma, chondrosarcoma, CHRPE, desmoid tumors, small intestine carcinoma, embryonal tumours, endometrial carcinoma, ependymoma, Fanconi anemia/medulloblastoma, thyroid follicular cell tumors, gastrointestinal carcinomas, gastrointestinal stromal tumors, glioblastoma, glioma, gonadoblastoma, hemangioblastomas of the retina and the CNS, breast hamartomas, hamartomas of the gastrointestinal tract, bladder carcinoma, skin carcinoma, skin tumors, hepatobiliary carcinomas, hepatoblastoma, hepatoma, hereditary leiomyomatosis, brain tumours, pituitary adenoma, keratoacanthomas, colon carcinoma, cutaneous melanoma, larynx carcinoma, leiomyosarcoma, skin leiomyoma, leukemia, Lhermitte-Duclos disease, liposarcoma, lymphoma, stomach and small intestine carcinomas, stomach carcinoma, diffuse type, malignant tumors of the gastrointestinal tract, malignant tumors of the urogenital tract, malignant melanoma, malignomas of the hematopoietic system, mammary carcinoma, mammary fibroadenoma, medullary thyroid carcinomas, medulloblastoma, medulloblastomas and other CNS tumors, melanoma, melanocytic skin lesions, meningioma, mesothelioma, multiple bilateral renal angiomyolipoma, myelodysplasia, myocardial rhabdomyoma, myxoid subcutaneous tumors, adrenocortical carcinoma, parathyroid adenomas, parathyroid hyperplasia, neurofibrosarcomas, renal pelvis carcinoma, kidney tumors (oncocytoma, chromophobe renal cell carcinoma, hybrid tumors), renal cell carcinoma, renal cell carcinoma-associated renal cell carcinoma, odontogenic keratocysts, optic gliomas, osteogenic sarcoma, osteoma, osteosarcoma, osteosarcoma, ovarian carcinoma, pancreatic islet cell tumors, pancreatic carcinoma, papillary renal cell carcinoma, papillary thyroid carcinoma, paraganglioma, pheochromocytoma, pheochromocytomas, squamous cell carcinoma, squamous cell carcinoma of the skin, squamous cell carcinomas, choroid plexus tumors, primary adrenocortical nodular hyperplasia, primitive neuroectodermal tumors, prostate carcinoma, retinoblastoma, rhabdoid tumors, rhabdomyosarcoma, giant cell astrocytoma, sarcoma/osteosarcoma, thyroid carcinoma, schwannoma, sebaceous adenomas, sebaceous epitheliomas, sebaceous carcinoma, testicular Sertoli cell tumor, testicular carcinoma, trichilemmomas of the skin, ureter carcinoma, uterine leiomyomas, vestibular schwannomas, atrial myxoma, soft tissue sarcoma, Wilms tumor and tongue carcinoma; preferably breast cancer, preferably triple-negative breast cancer, and pancreatic carcinoma. 24. A pharmaceutical composition comprising an inhibitor of acid lipase, preferably Lalistat or one of its physiologically acceptable salts, and also (i) a cell-toxic substance, (ii) a substance having an antitumor effect or (iii) immune cells directed against cancer cells. 25. The composition according to embodiment 24, wherein the (i) cell-toxic substance is selected from the group consisting of taxol, docetaxel, cisplatin, carboplatin, cytochalasin B, gramicidin D, ethidium bromide, emetine, mitomycin, etoposide, teniposide, vincristine, vinblastine, colchicine, doxorubicin, daunorubicin, dihydroxyanthracenedione, mitoxantrone, mithramycin, actinomycin, d,1-dihydrotestosterone, glucocorticoids, procaine, tetracaine, lidocaine, propranolol and puromycin; preferably taxol. 26. The composition according to embodiment 24, wherein the (ii) substance having an antitumor effect is selected from the group consisting of targeted active antitumor ingredients, active ingredients of anti-hormone therapy and immunostimulatory antibodies. The composition according to embodiment 24, wherein the (iii) immune cells directed against cancer cells are modified cytotoxic T cells.

The following examples elucidate the invention, but are not to be interpreted as restrictive:

### EXAMPLE 1

MDA-MB-231 cancer cells were incubated with Lalistat 2 (selective LAL inhibitor) or the formulation control (mock).

FIG. 1 shows the effects of acid lipase-inhibiting substances on cell proliferation in TNBC MDA-MB-231 cancer cells. As is evident from FIG. 1, the inhibition of the cellular lipase by means of Lalistat in MDA-MB-231 cancer cells leads to a distinct inhibition of cell proliferation compared to the formulation control.

The data depicted in FIG. 1 prove that, especially in the case of aggressive triple-negative breast cancer (TNBC), the use of Lalistat to pharmacologically address acid lipase has a high therapeutic potential to inhibit the cell growth of cancer cells.

### EXAMPLE 2

MDA-MB-231 cancer cells were incubated with Lalistat 2 (selective LAL inhibitor) or the formulation control (mock). For the formulation control, paclitaxel alone was used with the same concentration of DMSO as for Lalistat 2. 24 hours after seeding, the cancer cells were incubated for 3 days in 15% FCS-containing RPMI medium having concentrations of Lalistat descending from 50 μM or the corresponding DMSO concentration as control. Subsequently, CellTiterGlo® (Promega) was used to quantify the number of vital cancer cells.

PANC-1 cancer cells and MDA-MB-231 cancer cells were incubated with Lalistat 2 (selective LAL inhibitor) or the formulation control (mock) and paclitaxel (taxol). For the formulation control, paclitaxel alone was used with the same concentration of DMSO as for Lalistat 2. In a comparative experiment, the cancer cells were incubated with simvastatin (statin, HMG-CoA reductase inhibitor) or formulation control (mock) and paclitaxel (taxol) under otherwise identical conditions. 24 hours after seeding, the cancer cells were incubated for 6 days in 15% FCS-containing RPMI medium containing 50 μM Lalistat or 0.5 μM simvastatin and also a paclitaxel concentration of 80 nM to 40 μM. Subsequently, the viability of the cancer cells (cell viability) was determined using CellTiter-Glo® (Promega), a dose-effect curve was created and the ICso was calculated.

FIG. 2 shows the effects of acid lipase-inhibiting substances on the toxicity of paclitaxel in TNBC (A, B) MDA-MB-231 cancer cells and in pancreatic ductal adenocarcinoma (C) PANC-1 cancer cells. As is evident from FIG. 2, the inhibition of the cellular lipase by means of Lalistat/paclitaxel leads, in contrast to simvastatin/paclitaxel, to a reduction in the ICₛₒ value by approx. a factor of 4 in MDA-MB-231 cancer cells and approx. a factor of 3 in PANC-1 cancer cells compared to the formulation control. In contrast to simvastatin, Lalistat therefore distinctly increases the induction of cell death by paclitaxel, presumably via apoptosis.

Whereas statins for adjuvant hormone therapy of estrogen receptor-positive (ER-positive) breast cancer have been successfully tested in initial therapeutic approaches, no improvements in overall survival (OS) have been observed in initial studies for triple-negative breast cancer (TNBC) under statin treatment.

The data depicted in FIG. 2 prove that, especially in the case of aggressive triple-negative breast cancer (TNBC) or pancreatic ductal adenocarcinoma, the use of Lalistat in synergistic cancer therapies to pharmacologically address acid lipase has a significantly higher therapeutic potential than the inhibition of HMG-CoA reductase using statins.

### EXAMPLE 3

24 hours after seeding, MDA-MB-231 cancer cells were incubated for 6 days in 15% FCS-containing RPMI medium containing 50 μM Lalistat or formulation control (0.05% DMSO). Subsequently, the relative expression of the (A-C) tumor stem cell markers ALDH1, CD44 and the ratio CD44/CD24 and of the (D) mesenchymal marker vimentin was analyzed by means of quantitative PCR. The values were normalized to the housekeepers GAPDH or HRPT1. The target was amplified in a singleplex assay using “SYBR Green”.

FIG. 3 shows the relative change in expression of tumor stem cell markers and vimentin in MDA-MB-231 cancer cells after 6 days of treatment with Lalistat. The results of two independent experiments with three biological replicates in each case are depicted. Significance difference compared to formulation control: ALDH: 0.0026 (GAPDH), 0.0004 (HRPT1); CD44: >0.0001 (GAPDH/HRPT1); CD44/CD24: <0.01

As evidenced by the data in FIG. 3, the incubation of cancer cells with Lalistat leads to changes in epigenetics. What was demonstrated was a reduction in the expression of (tumor) stem cell markers and mesenchymal marker. As a result of the treatment with Lalistat, the cancer cells thus lose their (tumor) stem cell or mesenchymal character and thereby their tumorigenicity and malignancy. Since cell-endogenous defense mechanisms are reactivated in the course of re-differentiation, what can be expected in addition to the described apoptosis inducibility is, for example, also a higher immunogenicity of the cancer cells, which can be combined synergistically with immunotherapy. The inventors were able to demonstrate this for the first time via reduced PD-L1 expression on cancer cells after Lalistat treatment.

Further preferred embodiments (“AFs”) are the following:
AF 1: A method for finding inhibitors of acid lipase that are suitable for treatment of cancers, comprising the steps of
   (a) providing an inhibitor of acid lipase;
   (b) incubating cancer cells of a cancer cell line with the inhibitor of acid lipase and ascertaining a property of the cancer cells;
   (c) incubating cancer cells of the same cancer cell line as in step (b) in the absence of the inhibitor of acid lipase and ascertaining the same property of the cancer cells as in step (b) under the same conditions as in step (b);
   (d) comparing the ascertained property of the cancer cells according to steps (b) and (c).
AF2: The method according to AF1 for finding inhibitors of acid lipase that are suitable for adjuvant treatment of cancers, wherein step (b) is carried out in the presence of (i) a cell-toxic substance, (ii) a substance having an antitumor effect or (iii) immune cells directed against cancer cells; and wherein step (c) is carried out in the presence of the same substance or the same immune cells as in step (b).
AF3: The method according to AF 1 or AF2, wherein the property of the cancer cells that is ascertained is the survivability thereof.
AF4: The method according to AF2 or AF3, wherein
   the (i) cell-toxic substance is selected from the group consisting of taxol, docetaxel, cisplatin, carboplatin, cytochalasin B, gramicidin D, ethidium bromide, emetine, mitomycin, etoposide, teniposide, vincristine, vinblastine, colchicine, doxorubicin, daunorubicin, dihydroxyanthracenedione, mitoxantrone, mithramycin, actinomycin, d, 1-dihydrotestosterone, glucocorticoids, procaine, tetracaine, lidocaine, propranolol and puromycin; preferably taxol;
   the (ii) substance having an antitumor effect is selected from the group consisting of targeted active antitumor ingredients, active ingredients of anti-hormone therapy and immunostimulatory antibodies; or
   the (iii) immune cells directed against cancer cells are modified cytotoxic T cells.
AF5: The method according to any of the preceding AFs, wherein step (a) comprises the substeps of
   (a₁) providing multiple test substances;
   (a₂) screening the test substances for their inhibitory effect on the enzymatic activity of acid lipase;
   (a₃) selecting at least one screened test substance, the inhibitory effect of which is stronger than the inhibitory effect of at least one other screened test substance, and providing this selected test substance as an inhibitor of acid lipase.
AF6: The use of an inhibitor of acid lipase, preferably Lalistat or one of its physiologically acceptable salts, for production of a drug for treatment of a cancer.
AF7: The use according to AF6, wherein the treatment is effected as an adjuvant treatment in addition to a treatment (i) with a cell-toxic substance, (ii) with a substance having an antitumor effect or (iii) with immune cells directed against cancer cells.
AF8: The use according to AF7, wherein
   the (i) cell-toxic substance is selected from the group consisting of taxol, docetaxel, cisplatin, carboplatin, cytochalasin B, gramicidin D, ethidium bromide, emetine, mitomycin, etoposide, teniposide, vincristine, vinblastine, colchicine, doxorubicin, daunorubicin, dihydroxyanthracenedione, mitoxantrone, mithramycin, actinomycin, d, 1-dihydrotestosterone, glucocorticoids, procaine, tetracaine, lidocaine, propranolol and puromycin; preferably taxol;
   the (ii) substance having an antitumor effect is selected from the group consisting of targeted active antitumor ingredients, active ingredients of anti-hormone therapy and immunostimulatory antibodies; or
   the (iii) immune cells directed against cancer cells are modified cytotoxic T cells.
AF9: A pharmaceutical composition comprising an inhibitor of acid lipase, preferably Lalistat or one of its physiologically acceptable salts, and also (i) a cell-toxic substance, (ii) a substance having an antitumor effect or (iii) immune cells directed against cancer cells.
AF10: The composition according to AF9, wherein
   the (i) cell-toxic substance is selected from the group consisting of taxol, docetaxel, cisplatin, carboplatin, cytochalasin B, gramicidin D, ethidium bromide, emetine, mitomycin, etoposide, teniposide, vincristine, vinblastine, colchicine, doxorubicin, daunorubicin, dihydroxyanthracenedione, mitoxantrone, mithramycin, actinomycin, d, 1-dihydrotestosterone, glucocorticoids, procaine, tetracaine, lidocaine, propranolol and puromycin; preferably taxol;
   the (ii) substance having an antitumor effect is selected from the group consisting of targeted active antitumor ingredients, active ingredients of anti-hormone therapy and immunostimulatory antibodies; or
   the (iii) immune cells directed against cancer cells are modified cytotoxic T cells.

1. A method for finding inhibitors of acid lipase that are suitable for treatment of cancers, comprising the steps of:
(a) providing an inhibitor of acid lipase;
(b) incubating cancer cells of a cancer cell line with the inhibitor of acid lipase and ascertaining a property of the cancer cells;
(c) incubating cancer cells of the same cancer cell line as in step (b) in the absence of the inhibitor of acid lipase and ascertaining the same property of the cancer cells as in step (b) under the same conditions as in step (b);
(d) comparing the ascertained property of the cancer cells according to steps (b) and (c).

2. The method as claimed in claim 1 for finding inhibitors of acid lipase that are suitable for adjuvant treatment of cancers, wherein step (b) is carried out in the presence of (i) a cell-toxic substance, (ii) a substance having an antitumor effect, or (iii) immune cells directed against cancer cells; and wherein step (c) is carried out in the presence of the same substance or the same immune cells as in step (b).

3. The method as claimed in claim 1, wherein the property of the cancer cells that is ascertained is the survivability thereof

4. The method as claimed in claim 2, wherein:
the (i) cell-toxic substance is selected from the group consisting of taxol, docetaxel, cisplatin, carboplatin, cytochalasin B, gramicidin D, ethidium bromide, emetine, mitomycin, etoposide, teniposide, vincristine, vinblastine, colchicine, doxorubicin, daunorubicin, dihydroxyanthracenedione, mitoxantrone, mithramycin, actinomycin, d,1-dihydrotestosterone, glucocorticoids, procaine, tetracaine, lidocaine, propranolol, and puromycin; preferably taxol;
the (ii) substance having an antitumor effect is selected from the group consisting of targeted active antitumor ingredients, active ingredients of anti-hormone therapy and immunostimulatory antibodies; or
the (iii) immune cells directed against cancer cells are modified cytotoxic T cells.

5. The method as claimed in claim 1, wherein step (a) comprises the substeps of:
(a₁) providing multiple test substances;
(a₂) screening the test substances for their inhibitory effect on the enzymatic activity of acid lipase;
(a₃) selecting at least one screened test substance, the inhibitory effect of which is stronger than the inhibitory effect of at least one other screened test substance, and providing this selected test substance as an inhibitor of acid lipase.

6. A use of an inhibitor of acid lipase, preferably Lalistat or one of its physiologically acceptable salts, for production of a drug for treatment of a cancer, preferably triple-negative mammary carcinoma or tamoxifen-resistant hormone receptor-positive breast cancer, wherein the drug preferably contains a scavenger receptor B inhibitor, preferably BLT-1 (CAS No.: 321673-30-7), and/or a CD36 inhibitor.

7. The use as claimed in claim 6, wherein:
the treatment increases the apoptosis inducibility of the cancer cells; and/or
the treatment reduces the proliferation of the cancer cells; and/or
the treatment reduces the mesenchymal character and/or stem-cell character of the cancer cells; and/or
the treatment increases the immunogenicity of the cancer cells; and/or
the treatment (i) reduces or prevents the metastasis of the cancer cells and/or (ii) metastases-forming circulating tumor cells and/or cell clusters are killed and/or the formation of metastases therefrom is prevented; and/or
the cancer cells to be treated are tumor stem cells.

8. The use as claimed in claim 6 or 7, wherein the treatment is effected as an adjuvant treatment in addition to a treatment (i) with a cell-toxic substance, (ii) with a substance having an antitumor effect or (iii) with immune cells directed against cancer cells.

9. The use as claimed in claim 8, wherein:
the (i) cell-toxic substance is selected from the group consisting of taxol, docetaxel, cisplatin, carboplatin, cytochalasin B, gramicidin D, ethidium bromide, emetine, mitomycin, etoposide, teniposide, vincristine, vinblastine, colchicine, doxorubicin, daunorubicin, dihydroxyanthracenedione, mitoxantrone, mithramycin, actinomycin, d,1-dihydrotestosterone, glucocorticoids, procaine, tetracaine, lidocaine, propranolol, and puromycin; preferably taxol;
the (ii) substance having an antitumor effect is selected from the group consisting of targeted active antitumor ingredients, active ingredients of anti-hormone therapy and immunostimulatory antibodies; or
the (iii) immune cells directed against cancer cells are modified cytotoxic T cells.

10. A pharmaceutical composition comprising an inhibitor of acid lipase, preferably Lalistat or one of its physiologically acceptable salts, and also (i) a cell-toxic substance, (ii) a substance having an antitumor effect or (iii) immune cells directed against cancer cells.

11. The composition as claimed in claim 10, wherein:
the (i) cell-toxic substance is selected from the group consisting of taxol, docetaxel, cisplatin, carboplatin, cytochalasin B, gramicidin D, ethidium bromide, emetine, mitomycin, etoposide, teniposide, vincristine, vinblastine, colchicine, doxorubicin, daunorubicin, dihydroxyanthracenedione, mitoxantrone, mithramycin, actinomycin, d,1-dihydrotestosterone, glucocorticoids, procaine, tetracaine, lidocaine, propranolol, and puromycin; preferably taxol;
the (ii) substance having an antitumor effect is selected from the group consisting of targeted active antitumor ingredients, active ingredients of anti-hormone therapy and immunostimulatory antibodies; or
the (iii) immune cells directed against cancer cells are modified cytotoxic T cells.

12. The composition as claimed in claim 10, which is prepared for treatment of a cancer, preferably prepared for treatment of triple-negative mammary carcinoma or tamoxifen-resistant hormone receptor-positive breast cancer.

13. The composition as claimed in claim 10 for use in the treatment of a cancer, preferably for use in the treatment of triple-negative mammary carcinoma or tamoxifen-resistant hormone receptor-positive breast cancer.

14. A use of the composition as claimed in claim 10 for production of a drug for treatment of a cancer, preferably for production of a drug for treatment of triple-negative mammary carcinoma or tamoxifen-resistant hormone receptor-positive breast cancer.
